(19) United States
(12) Patent Application Publication
(10) Pub. No.: US 2015/0182201 A1
(43) Pub. Date: Jul. 2, 2015

(54) ULTRASOUND PROBE POWER SUPPLY

(71) Applicant: General Electric Company, Schenectady, NY (US)

(72) Inventors: Franz Josef Steinbacher, Pfaffing (AT); Andreas Kremsl, St. Wolfgang (AT)

(73) Assignee: General Electric Company, Schenectady, NY (US)

(21) Appl. No.: 14/145,517

(22) Filed: Dec. 31, 2013

(51) Int. Cl. A61B 8/00; G01R 21/06

(52) U.S. Cl. CPC A61B 8/56; G01R 21/06; A61B 8/4444

(57) ABSTRACT

A power supply supplies power to an ultrasound probe across a cable by determining an output current at a power supply source supplying power to the ultrasound probe across the cable, determining a voltage drop across the cable based upon the determined output current and regulating an output voltage of the power supply source based on the determined voltage drop.

### BACKGROUND

Ultrasound or ultrasonography is a medical imaging technique that utilizes high-frequency (ultrasound) waves and their reflections. Such ultrasound waves are directed into a person's anatomy using a handheld or portable probe. The probe senses reflections or echoes of the ultrasound waves and transmits the sensed echoes to a separate host unit which produces an image of the anatomy based upon the sensed echoes. Many probes include active electronics, such as a beam former and amplifiers, rendering such probes extremely sensitive to variations in power supplied to the probe. Variations in the power supplied to the probe may result in visible artifacts in the image.

### BRIEF DESCRIPTION OF THE DRAWINGS

FIG. 1 is a schematic diagram of an example ultrasound diagnostic system.

FIG. 2 is a flow diagram of an example method that may be carried out by the system of FIG. 1.

FIG. 3 is a schematic diagram of an example ultrasound probe of the system of FIG. 1.

FIG. 4 is a schematic diagram of an example ultrasound probe power supply of the system of FIG. 1.

FIG. 5 is a circuit diagram of an example implementation of the ultrasound diagnostic system of FIG. 1.

### DETAILED DESCRIPTION OF THE EXAMPLE EMBODIMENTS

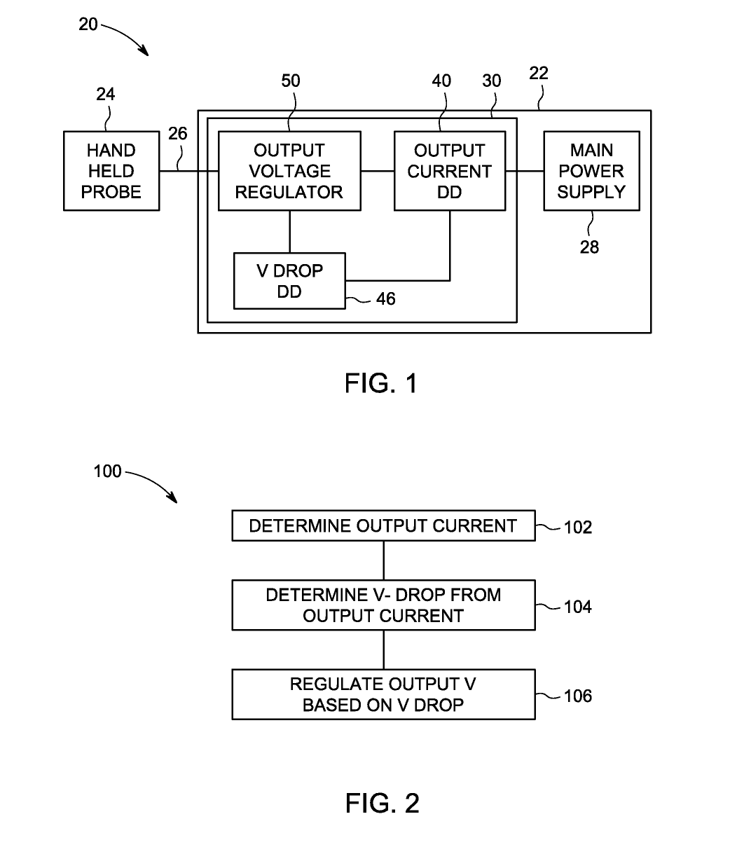

FIG. 1 schematically illustrates an example ultrasound diagnostic system 20 which comprises a main or host unit 22 that supplies power to a handheld or portable ultrasound transducer or probe 24 across a power supply cable 26. As will be described hereafter, system 20 supplies power to probe 24 across cable 26 in a stable manner, increasing the performance of probe 24 and reducing the presence of visible artifacts in images generated based upon signals produced by probe 24.

Probe 24 comprises a handheld or portable unit configured to direct high-frequency ultrasound waves at an anatomy and to capture receive reflections of such waves to produce signals which are used to generate an image of the anatomy. Probe 24 comprises electronics configured to operate at a nominal predetermined voltage. In one implementation, probe 24 includes electronics comprising amplifiers, such as low noise amplifiers. In one implementation, probe 24 includes electronics comprising amplifiers and beam formers. In other implementations, some of the electronics, such as beam formers, are provided as part of host unit 22.

Host unit 22 comprises a base unit or main unit configured to supply power to probe 24 across cable 26. Host unit 22 comprises main power supply 28 and transducer or probe power supply 30. Although not illustrated, in some implementations, host unit 22 may additionally comprise other components such as a processing componentry for generating an image based upon the signals received from probe 24, a display for presenting the generated image and a user interface by which a person may control the operation of system 20.

Main power supply 28 comprises a source of general power to the various electronic components of system 20. Probe power supply 30 adjusts and regulates the power receive from main power supply 28 for use by one or more electronic components of probe 24. Probe power supply 30 provides power having an output voltage that is conducted to probe 24 across cable 26. In many instances, the long length of cable 26 results in significant voltage drop on cable 26. This may result in the voltage received at probe 24 being unstable, reducing the performance of system 20 and the quality of images produced by system 20. To provide a more stable voltage at probe 24 and enhance the performance of system 20, probe power supply 30 regulates the output voltage of the power at power supply 30 that is to be conducted to probe 24 across cable 26 so as to dynamically compensate for voltage drop that occurs across cable 26.

Probe power supply 30 comprises output current determining device 40, voltage drop determining device 46 and output voltage regulator 50. Output current determining device 40 comprises a device, such as electrical circuitry, configured to determine the output current of power supply 30. In one implementation, output current determining device 40 comprises an electrical current sensing shunt. In other implementations, output current determining device 40 may comprise other current determining devices or circuitry.

Voltage drop determining device 46 comprises a device, such as electrical circuitry, configured to detect and determine, estimate or determine the voltage drop currently occurring across cable 26 based upon signals received from the output current determining device 40 indicating the determined output current. The voltage drop across cable 26 is a function of the resistance of cable 26 and the present electrical current of the power being transmitted across cable 26. In the example illustrated, voltage drop determining device 46 determines the voltage drop across cable 26 by multiplying the determined output current received from device 40 by a constant k which is based upon a nominal electrical resistance of cable 26. In other implementations, voltage drop determining device 46 may determine, determine or estimate the voltage drop across cable 26 based upon other parameters or in other manners.

Output voltage regulator 50 comprises a device, such as electrical circuitry, configured to adjust, control and regulate the output voltage of power supply 30 based upon the determined, or estimated voltage drop across 26. Output voltage regulator 50 adjusts, controls and regulates the output voltage of power supply 30 based upon signals received from device 46 indicating the determined voltage drop across cable 26. In the example illustrated, output voltage regulator 50 regulates the voltage of the power being output by power supply 30 based upon the determined output current such that the power has a voltage greater than the nominal or operating voltage of the electronics of probe 24 that are being supplied power by power supply 30. In the example illustrated, output voltage regulator 50 regulates a voltage of the power being output by power supply 30 based upon the determined output current such that the power has a voltage corresponding to or approximating the nominal or operating voltage of the electronics of probe 24 that are being supplied power by power supply 30 and/plus the determined voltage drop across cable 26. As a result, after experiencing the voltage drop that occurs across cable 26, the power received at probe 24 is at or more near the nominal voltage for which the electronics of probe 24 are designed to operate.

FIG. 2 is a flow diagram of an example method 100 that may be carried out by system 20. As indicated by step 102, output current determining device 40 determines the output current of the power being output by power supply 30 to cable 26 and ultimately to the assigned electronics of probe 24. As noted above, in one implementation, the output current is determined by electronic circuitry such as a shunt which outputs signals representing the determined current.

As indicated by step 104, voltage drop determining device 46 determines a voltage drop across cable 26 (and any additional non-load resistances between power supply 30 and the consumer electronics of probe 24). For purposes of this disclosure, the term of “determining” or “determine” includes calculating and/or estimating. The voltage drop is determined based upon signals received from determining device 40 indicating the determined output current of power supply 30. In one implementation, the voltage drop is determined based upon the determined output current of power supply 30 and a predetermined constant that is based upon the electrical resistance of cable 26. In some implementations, the constant is based upon resistances of other components or based upon other factors as well.

As indicated by step 106, output voltage regulator 50 adjusts, regulates or controls the output voltage of power supply 30 based upon the signal received from device 46 indicating the determined voltage drop across cable 26. As discussed above with respect to system 20, in one implementation, output voltage regulator 50 regulates the voltage of the power being output by power supply 30 such that the power has a voltage greater than the nominal or operating voltage of the electronics of probe 24 that are being supplied power by power supply 30. In one implementation, output voltage regulator 50 regulates a voltage of the power being output by power supply 30 such that the power has a target voltage corresponding to an amount equal to the nominal or operating voltage of the electronics of probe 24 that are being supplied power by power supply 30 and an additional voltage based upon the determined voltage drop across cable ii26. As a result, after experiencing the voltage drop that occurs across cable 26, the power received at probe 24 is at or more near the nominal voltage for which the electronics of probe 24 are designed to operate.

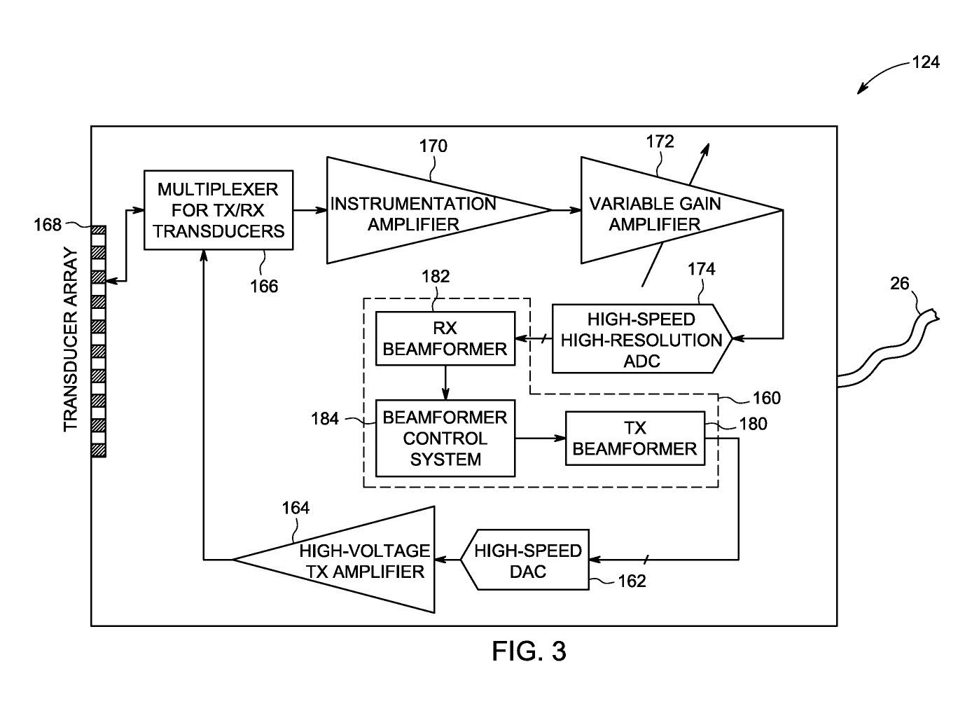

FIG. 3 schematically illustrates handheld or portable probe 124, an example implementation of probe 24 for system 20 shown in FIG. 1. In the example shown in FIG. 3, probe 124 comprises beam former system 160, digital to analog converter 162, amplifiers 164, multiplexer 166, transducer array 168, amplifiers 170, variable gain amplifiers 172 and analog-to-digital converter 174. Beam former system 160 comprises electronic circuitry that controls the operation of transducer array 168. Beam former system 160 comprises a digital transmit (TX) beam former 180, a receiver (RX) beam former 182 and a control system 184. Transmit beam former 180, under the control of controller 184, directs the output and receipt of ultrasound waves in one or more directions using one or more algorithms that control transducer array 168 to form a wave front that generates constructive interference.

Digital to analog converter 162 converts to digital signals from beam former 160 to analog signals for the control of transducer array 168. Amplifier 164 amplifies the analog electrical signals received from converter 162 and transmit such signals to multiplexer 166. Multiplexer 166 transmits the amplified electrical signals to the transducer elements of transducer array 168 for output to the patient's anatomy. Transducer array 168 generates high-frequency ultrasound waves in response to or based upon the amplified electrical signals received from multiplexer 166. In one implementation, transducer array 168 comprises a large array of piezoelectric crystals which vibrate or change shape at high frequencies to produce the ultrasound waves. In response to being impacted by sound or pressure waves, the piezoelectric crystals produce electrical currents which are transmitted to multiplexer 166.

Amplifiers 170 and 172 amplify the analog electrical signals output or produced by the transducer elements of array 168 and forwarded by multiplexor 166. In one implementation, the amplifiers comprise low noise amplifiers. Analog-to-digital converter 174 converts the amplified analog electrical signals originating from the elements of transducer array 168 into digital signals which are transmitted to receive beam former 182. Receive beam former 182, using one or more algorithms and under the control of control system 184, associates a directional component to the digital signals representing the echoes from the patient's anatomy. The resulting signals are transmitted to host unit 22 for analysis and/or display of an image.

In the example illustrated, the operation of amplifiers 164, 170, 172, as well as the operation of the components of beam former 160 are highly sensitive to the voltage supplied across cable 26 from power supply 30 (shown in FIG. 1). Because power supply 30 automatically compensates for the voltage drop across cable 26, the power supplied to and received at beam former 160 and amplifiers 164, 170, 172 has a more stable voltage, resulting in improved performance. In one implementation, power supply 30 provides power to probe 124 at or within a range of +/−5% of the nominal predetermined operating voltage of the electronic components of probe 124, such as beam former 160 and/or amplifiers 164, 170, 172. In some implementations, beam former 160 is alternatively provided as part of host unit 22, wherein the digital signals output by beam former 160 are transmitted in a wired or wireless fashion to probe 124.

In other implementations, probe 124 alternatively comprises an analogue receive (Rx) beam former without an analog-to-digital converter, wherein the output of the beam former is transmitted as an analogue signal to the host over the cable 26. In other implementations, probe 124 may alternatively employ an analogue transmit (Tx) pulser in place of the illustrated transmit (Tx) beam former 180, digital to analog converter 162 and high-voltage transmit amplifier 164. In yet other implementations, probe 124 may have other configurations.

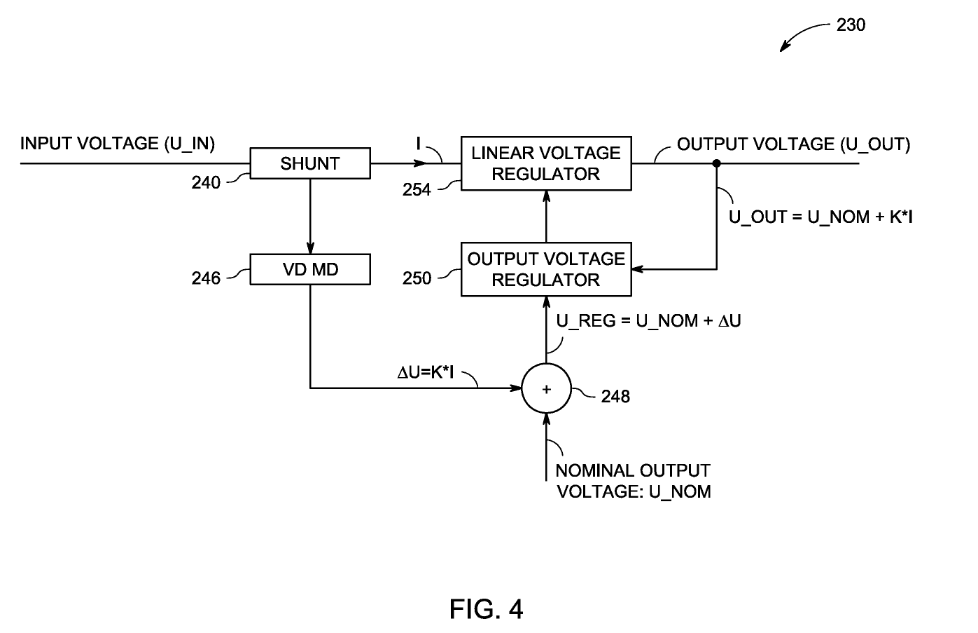

FIG. 4 schematically illustrates power supply 230, an example implementation of power supply 30 of system 20 shown in FIG. 1. Power supply 230 comprises shunt 240, voltage drop determining device 246, adder 248, output voltage regulator 250 and linear voltage regulator 254. Shunt 240 comprises a device configured to detect the output current of power supply 230. In one implementation, shunt 240 comprises a manganin resistor of an accurately known resistance, wherein the voltage drop across shunt 240 indicates current flow. In other implementations, shunt 240 may have other constructions. Shunt 240 outputs signals indicating the output current to voltage drop determining device 246.

Voltage drop determining device 246 receives the determined output current and determines the voltage drop across cable 26 based upon the output current. In the example illustrated, voltage drop determining device 246 determines the voltage drop ΔU by multiplying the determined output current I by a predetermined constant k based upon a predetermined electrical resistance of cable 26. Adder 248 ads a signal indicating the voltage drop voltage value ΔU to the nominal voltage Uₙₒₘ.

Output voltage regulator 250 utilizes the determined voltage to be output by device supply 30, as received from adder 248, to regulate or control the voltage being output by power supply 30. As shown FIG. 4, regulator 250 receives feedback of the actual output voltage after being modified by linear voltage regulator 254. Such feedback assists in the regulation or control of the output voltage by regulator 250.

Linear voltage regulator 254 maintains a steady output voltage for power supply 30. In one implementation, linear voltage regulator 254 comprises a low-dropout regulator (LDO). In some implementations, other electrical circuits or components may use to facilitate a steady output voltage for power supply 30.

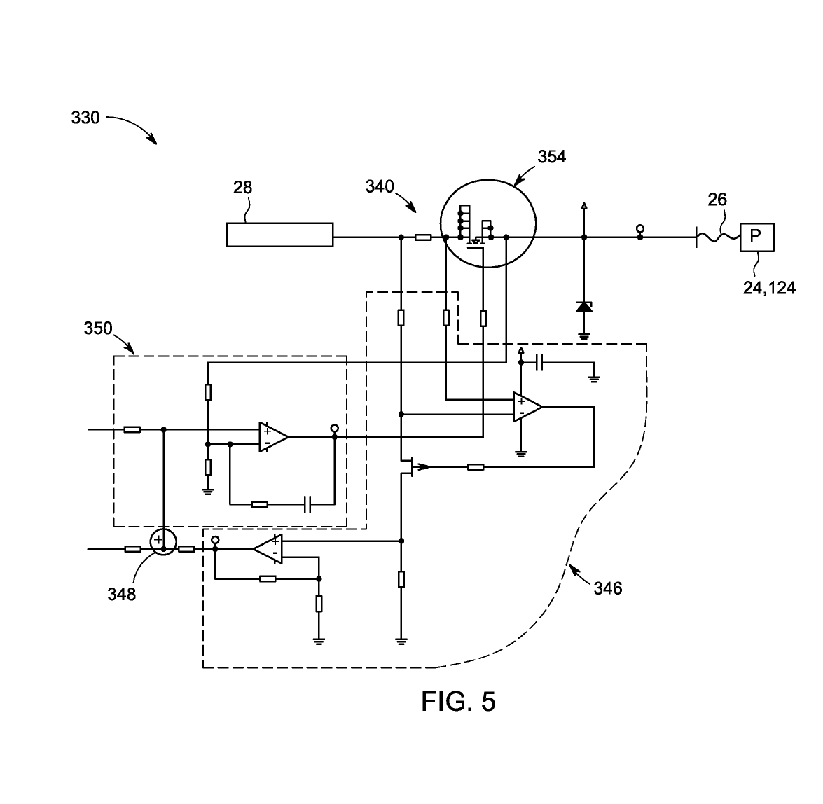

FIG. 5 is a circuit diagram illustrating power supply 330, another example implementation of power supply 30. FIG. 5 illustrates output current determining device 340, in the form of an electrical current determining shunt, voltage drop determining device 346 implemented with electrical circuitry, adder 348 providing a signal indicating the nominal voltage to be supplied at the assigned electronics of probe 24 (such as beam former 160 and amplifiers 162, 164, 172 of probe 124) by power supply 330, output voltage regulator 350, implemented with electrical circuitry, and linear voltage regulator 354 implemented as a low-dropout regulator. In other implementations, one or more of such components may alternatively be implemented with circuitry as well as one or more processing units following instructions stored are contained on one or more non-transitory computer-readable mediums.

Although the present disclosure has been described with reference to example embodiments, workers skilled in the art will recognize that changes may be made in form and detail without departing from the spirit and scope of the claimed subject matter. For example, although different example embodiments may have been described as including one or more features providing one or more benefits, it is contemplated that the described features may be interchanged with one another or alternatively be combined with one another in the described example embodiments or in other alternative embodiments. Because the technology of the present disclosure is relatively complex, not all changes in the technology are foreseeable. The present disclosure described with reference to the example embodiments and set forth in the following claims is manifestly intended to be as broad as possible. For example, unless specifically otherwise noted, the claims reciting a single particular element also encompass a plurality of such particular elements.

What is claimed is:

1. An ultrasound diagnostic apparatus comprising:
a power supply to be connected to an ultrasound probe by a cable, the power supply comprising:
an output current determining device to determine output current to the cable;
a voltage drop determining device to determine a voltage drop across the cable based on the determined output current; and
an output voltage regulator to regulate output voltage to the cable based on the determined voltage drop.

2. The ultrasound diagnostic apparatus of claim 1 further comprising the cable and the probe, the probe comprising a beam former and low noise amplifiers, wherein the beam former and low noise amplifiers are to receive power from the regulated output voltage transmitted across the cable.

3. The ultrasound diagnostic apparatus of claim 2, wherein the beam former and the low noise amplifiers are configured to operate at a nominal voltage and wherein the output voltage is regulated so as to correspond to the determined voltage drop added to the nominal voltage.

4. The ultrasound diagnostic apparatus of claim 1, wherein the output current determining device comprises a shunt.

5. The ultrasound diagnostic apparatus of claim 1 further comprising a linear voltage regulator between the output voltage regulator and the cable.

6. The ultrasound diagnostic apparatus of claim 4, wherein the linear voltage regulator comprises a low dropout regulator.

7. The ultrasound diagnostic apparatus of claim 1, wherein the output voltage is regulated so as to correspond to the determined voltage drop added to a nominal voltage to be supplied to the probe absent any voltage drop across the cable.

8. A method for supplying power to an ultrasound probe across a cable, the method comprising:
determining an output current at a power supply source supplying power to the ultrasound probe across the cable;
determining a voltage drop across the cable based upon the determined output current;
regulating an output voltage of the power supply source based on the determined voltage drop.

9. The method of claim 8, wherein the output voltage a voltage corresponding to the determined voltage drop added to a nominal voltage to be supplied to the probe absent any voltage drop across the cable.

10. The method of claim 8 further comprising outputting power to the cable at the output voltage, wherein the cable supplies the power to a beam former and low noise amplifiers of the probe.

11. The method of claim 8, wherein the output current at the power supply sources determined using a shunt.

12. The method of claim 8 further comprising regulating the output voltage with a linear voltage regulator.

13. The method of claim 12, wherein the linear voltage regulator comprises a low dropout regulator.

14. An ultrasound diagnostic apparatus comprising:
an ultrasound probe comprising a beam former and low noise amplifiers, wherein the beam former and low noise amplifiers are configured for operation at a nominal voltage;
a power cable having an electrically conductive line to supply power to the beam former and low noise amplifiers; and
a power supply to supply power to the electric conductive line of the power cable at an output voltage, the power supply comprising:
an output current determining device to determine output current to the cable;
a voltage drop determining device to determine a voltage drop across the cable based on the determined output current; and
an output voltage regulator to regulate output voltage to the cable based on the determined voltage drop such that the beam former and low noise amplifiers receive power at the nominal voltage.

15. The ultrasound diagnostic apparatus of claim 14, wherein the output current determining device comprises a shunt.

16. The ultrasound diagnostic apparatus of claim 14 further comprising a linear voltage regulator between the output voltage regulator and the cable.

17. The ultrasound diagnostic apparatus of claim 18, wherein the linear voltage regulator comprises a low dropout regulator.

18. The ultrasound diagnostic apparatus of claim 14, wherein the output voltage is regulated so as to correspond to the determined voltage drop added to a nominal voltage to be supplied to the probe absent any voltage drop across the cable.
